(19) United States
(12) Patent Application Publication
(10) Pub. No.: US 2024/0066205 A1
(43) Pub. Date: Feb. 29, 2024

(54) CT DEVICE AND METHOD FOR CONTROLLING CT DEVICE

(71) Applicant: SHANGHAI UNITED IMAGING HEALTHCARE CO., LTD., Shanghai (CN)

(72) Inventors: KUO-YUE LIU, Shanghai (CN); LONG YANG, Shanghai (CN)

(21) Appl. No.: 18/238,373

(22) Filed: Aug. 25, 2023

(30) Foreign Application Priority Data
Aug. 25, 2022 (CN) 202211036949.8

(51) Int. Cl. A61B 6/03; A61M 5/14; A61M 5/168; A61M 25/02

(52) U.S. Cl. CPC A61B 6/035; A61M 5/1417; A61M 5/1418; A61M 5/16854; A61M 5/16886; A61M 2025/024; A61M 2205/103

(57) ABSTRACT

The present disclosure provides a CT device and a method for controlling a CT device. The CT device includes a CT gantry and at least one infusion clip. The CT gantry includes a first end cover and a second end cover opposite to each other. Either or both of a surface of the first end cover and a surface of the second end cover is provided with a guiding rail. One end of each infusion clip is connected to one guiding rail and is slidable along the guiding rail, and the other end of the infusion clip is configured to position an infusion assembly.

### CROSS-REFERENCE TO RELATED APPLICATIONS

This application claims priority of Chinese Patent Application No. 202211036949.8, filed on Aug. 25, 2022, entitled “CT DEVICE AND METHOD FOR CONTROLLING CT DEVICE”, the content of which is hereby incorporated by reference in its entirety.

### TECHNICAL FIELD

The present disclosure relates to medical devices, and in particular to a CT device and a method for controlling a CT device.

### BACKGROUND

Currently, infusion stands for patients during computer tomography (CT) scans have no unified standard. Generally, third-party accessories are used in such cases. According to their arrangement methods, the infusion stands can be classified into the bed-end integrated type, the free-standing mobile type, and the ceiling-hung type.

The bed-end integrated type infusion stand is assembled at the end of a scanning bed, which requires a long infusion catheter during the scan. In addition, during a contrast-enhanced scan, the high-pressure injector is usually placed adjacent to a CT gantry, which prevents the infusion catheter from being fixed to the infusion stand. The bed-end integrated type infusion stand also has the problem of interference with the CT gantry, especially when the CT gantry is tilted forward or backward at a certain angle, the infusion stand may reach the CT gantry, so that the position of the infusion stand is greatly restricted.

The ceiling-hung type infusion stand is fixed to the ceiling of the scanning room. Although the ceiling-hung type infusion stand allows for a certain range of movement, the range of movement cannot be adjusted. In addition, it can be inconvenient to hang an infusion bag on the high-positioned infusion stand, making the process of hanging the infusion bag cumbersome. Moreover, the problem of interference with the infusion stand will also be occurred when the CT gantry is tilted forward and backward.

The free-standing mobile type infusion stand is primarily a third-party accessory, which is typically placed on a side of the scanning bed. When the bed slides into the gantry, the lengthy infusion catheter may be trapped by the scanning bed or piled up in the CT scan field, which affects CT image quality. In addition, the additional infusion stand also affects the layout in the scanning room.

The above three types all have limitations, and are restricted by the patient's scanning position and the length of the infusion catheter. Most manufactures utilize the integrated type infusion stand, fixing the infusion stand to the side and the end of the bed. According to relevant regulations, it is necessary to prevent hands from being pinched. A minimum gap between the CT gantry and the infusion stand should be greater than 25 mm. Therefore, especially for a head-first position of a CT scan, the bed-end integrated type infusion stand will drag a long infusion catheter, which is very inconvenient.

### SUMMARY

Accordingly, a CT device and a method for controlling a CT device are provided.

A CT device includes a CT gantry and at least one infusion clip. The CT gantry is provided with a guiding rail. One end of each infusion clip is connected to one guiding rail, and the other end of the infusion clip is configured to position an infusion assembly.

In some embodiments, the infusion assembly is used for infusion to the patient, and includes an infusion bag and at least a part of an infusion catheter. In the CT device, the CT gantry is also used as an infusion stand, and the guiding rail is arranged on the CT gantry, so that the infusion assembly can be hung adjacent to the gantry aperture of the CT device, and the infusion assembly fixed to the CT gantry will not interfere with other devices, simplifying the overall structure. In addition, since the infusion clip for fixing the infusion assembly is slidably connected to the guiding rail, when a scanning bed moves, a length of the infusion catheter of the infusion assembly outside the guiding rail can be adjusted by moving the infusion clip along the guiding rail, so that the infusion catheter will not be wound, stacked or stretched between the scanning bed and the CT gantry, so as to meet the infusion requirements of patients in different scanning scenarios.

In some embodiments, the CT device according further includes a hook connected to an upper portion of the CT gantry. The infusion assembly comprises an infusion bag and an infusion catheter, the hook is configured to hang the infusion bag, and the infusion clip is configured to fix the infusion catheter.

In some embodiments, the infusion bag is hung on the hook, and the infusion catheter is fixed to the infusion clip, the load carried by the infusion clip can be reduced. When the infusion clip moves, the infusion clip only drives the infusion catheter to move without the need to drive the infusion bag to move, which can reduce the weight during transportation and allows for smoother sliding of the infusion clip along the guiding rail.

In some embodiments, the guiding rail is a locking groove defined by the CT gantry, the infusion clip comprises a main body and a collar portion located at an end of the main body, the collar portion is locked in the locking groove, the main body defines a clamping hole, and the clamping hole is configured to allow the infusion catheter to extend through and to be fixed in the clamping hole.

In some embodiments, the locking groove a is recessed into the CT gantry, which occupies less space and has a more compact structure than a protruding guiding rail. The locking groove a is also convenient for the infusion clip to be mounted in the locking groove a for easy disassembly and assembly. In other embodiments, the guiding rail may be configured as a protruding track.

In some embodiments, the CT device further includes a pressure sensor mounted in the clamping hole and abutting against the infusion catheter, and the pressure sensor is configured to monitor a pressure in the infusion catheter.

In some embodiments, the pressure sensor can be electrically connected to a display device which is configured to display a pressure value, or the pressure sensor can be electrically connected to an alarm device. When the pressure value in the infusion catheter exceeds a threshold, the pressure sensor displays the pressure value through the display device or gives an alarm through the alarm device, as an early warning to alert the doctor and the patient. Especially when contrast medium or other medicinal liquid spills out from a blood vessel, the pressure in the infusion catheter increases, and the pressure sensor can provide an early warning.

In some embodiments, the CT device further includes a flow rate sensor mounted in the clamping hole and abutting against the infusion catheter, and the flow rate sensor being configured to monitor a flow rate of liquid in the infusion catheter.

In some embodiments, the flow rate sensor can detect a flow rate of medicinal solution in the infusion catheter in real time, and the flow rate of medicinal solution can provide a basis for the doctor to evaluate a status of the patient in real time. Especially for patients with poor heart function, the status of the patient can be evaluated in real time through real-time detection of the flow rate of the medicinal solution.

In some embodiments, the CT device further includes a stepper motor axially connected to the infusion clip and a controller electrically connected to the stepper motor. The controller is configured to control the stepper motor to rotate, thereby driving the infusion clip to move along the guiding rail.

In some embodiments, the controller controls the stepper motor to rotate, upon which the infusion clip is driven to move along the guiding rail, so as to achieve the precise retraction and release of the infusion catheter. Since the patient's inspection portion can be known in advance, information of the patient's inspection portion can be input into the controller in advance. When the controller controls the scanning bed to move, the controller also controls the stepper motor to rotate, so that retraction and release of the infusion catheter can be in correspondence with the movement of the scanning bed, thereby achieving an automatic adjustment of the infusion catheter.

In some embodiments, the CT device further includes a rack disposed adjacent to and parallel with the guiding rail and a pinion mounted to an output shaft of the stepper motor and engaged with the rack.

In some embodiments, the stepper motor drives the pinion to roll on the rack, driving the stepper motor to move along the locking groove a. Since the stepper motor is axially connected to the infusion clip, when the stepper motor moves, the infusion clip is driven to move accordingly. The transmission mode of the pinion and the rack is convenient for the infusion clip to move back and forth.

In some embodiments, the guiding rail is in an arc-shaped structure which is concave downward, or the guiding rail is arranged in a straight structure.

In some embodiments, the guiding rail with the arc shape is beneficial to cooperate with the CT gantry, and a relatively long guiding rail can be arranged on the CT gantry, so that a moving distance of the infusion flip on the guiding rail can be increased. the guiding rail with a straight structure facilitates the manufacture of the guiding rail.

In some embodiments, the infusion clip is rotatably connected to the guiding rail.

In the present embodiment, the above-mentioned structural is adopted to meet the requirement of adjusting the arrangement direction of the infusion catheter.

In some embodiments, the infusion clip is made of elastic material.

In some embodiments, the at least one infusion clip is a plurality of infusion clips, arranged on the guiding rail.

In the present embodiment, the infusion clip is made of elastic material, which facilitate the assembly and disassembly of the infusion clip. A plurality of infusion clips are arranged on the guiding rail, which improves the fixing effect.

In some embodiments, the CT gantry includes a first end cover and a second end cover opposite to each other, either or both of a surface of the first end cover and a surface of the second end cover is provided with the guiding rail.

A method for controlling the aforementioned CT device includes: driving the infusion clip to move along the guiding rail, such that a movement of the infusion clip is corresponding to a movement of the scanning bed from outside a gantry aperture of the CT device into the gantry aperture or a movement of the scanning bed from the gantry aperture to outside the gantry aperture.

In some embodiments, when the scanning bed moves from the outside towards the gantry aperture of the CT device to reach the position of a laser light of the CT device, the infusion site of the patient on the scanning bed gradually approaches the lowest end of the guiding rail, and thus the length of the infusion catheter will become redundant. During this period, the controller controls the stepper motor to rotate in a first direction, thereby driving the infusion clip to move down along the guiding rail, so as to retract the redundant infusion catheter between the patient's infusion site and the CT gantry in the guiding rail, preventing the infusion catheter from being twisted and messy.

In some embodiments, When the scanning bed continues to move from the position of the laser light toward the gantry aperture, the infusion site of the patient on the scanning bed gradually departs from the lowest end of the guiding rail, and the controller controls the stepper motor to rotate in a second direction opposite to the first direction, thereby driving the infusion clip to move upward along the guiding rail, so as to release the infusion catheter to meet the distance requirement between the patient's infusion site and the CT gantry, and the infusion catheter is prevented from being tightened or falling off, which causes safety hazards.

In some embodiments, conversely, when the scanning bed moves from inside the gantry aperture of the CT device, passes the laser light, and gradually outside the gantry aperture, the stepper motor causes the infusion clip to first retract and then release the infusion catheter.

On the basis of conforming to common knowledge in the field, the above-mentioned features can be combined arbitrarily.

In some embodiments, the infusion assembly is used for infusion to the patient, and includes an infusion bag and at least a part of an infusion catheter. In the CT device, the CT gantry is also used as an infusion stand, and the guiding rail is arranged on the CT gantry, so that the infusion assembly can be hung adjacent to the gantry aperture of the CT device, and the infusion assembly fixed to the CT gantry will not interfere with other devices, simplifying the overall structure. In addition, since the infusion clip for fixing the infusion assembly is slidably connected to the guiding rail, when a scanning bed moves, a length of the infusion catheter of the infusion assembly outside the guiding rail can be adjusted by moving the infusion clip along the guiding rail, so that the infusion catheter will not be wound, stacked or stretched between the scanning bed and the CT gantry, so as to meet the infusion requirements of patients in different scanning scenarios.

### BRIEF DESCRIPTION OF THE DRAWINGS

FIG. 1 is a schematic view of a CT device according to an embodiment of the present disclosure.

FIG. 2 is a schematic view of an infusion clip according to an embodiment of the present disclosure.

FIG. 3 is a schematic cross-sectional view of an infusion clip according to an embodiment of the present disclosure.

FIG. 4 is a schematic cross-sectional view of an infusion clip according to another embodiment of the present disclosure.

FIG. 5 is a schematic partial cross-sectional view of a CT device according to an embodiment of the present disclosure.

FIG. 6 is a front view of a CT device according to an embodiment of the present disclosure.

FIG. 7 is a side view of a CT device according to an embodiment of the present disclosure.

FIG. 8 is a schematic view of a CT device according to another embodiment of the present disclosure.

FIG. 9 is a flowchart of a method for controlling a CT device according to an embodiment of the present disclosure.

FIG. 10 is a schematic partial cross-sectional view of a CT device according to another embodiment of the present disclosure.

FIG. 11 is a schematic view of a CT device according to another embodiment of the present disclosure.

FIG. 12 is a flowchart of a method for controlling a CT device according to another embodiment of the present disclosure.

### DETAILED DESCRIPTION OF THE EMBODIMENTS

Embodiments of the present disclosure are described more fully hereinafter with reference to the accompanying drawings. The various embodiments of the present disclosure may, however, be embodied in many different forms and should not be construed as limited to the embodiments set forth herein. Rather, these embodiments are provided so that this disclosure will be thorough and complete, and will fully convey the scope of the present disclosure to those skilled in the art. Elements that are identified using the same or similar reference characters refer to the same or similar elements.

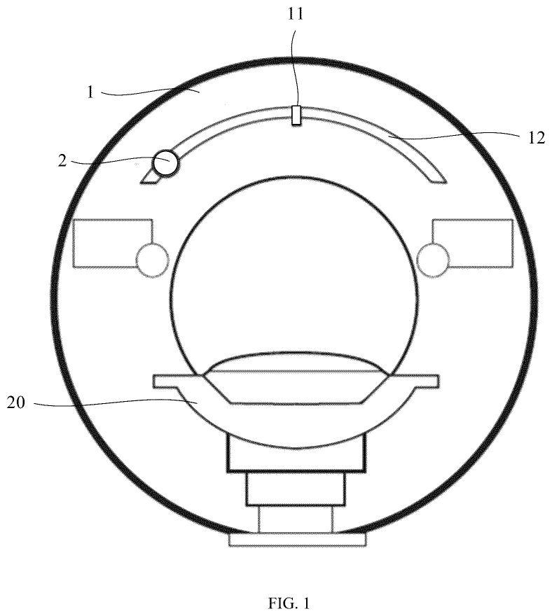

Referring to FIGS. 1 to 6, a CT device is provided according to an embodiment of the present disclosure. The CT device includes a CT gantry 1 and an infusion clip 2. The CT gantry 1 includes a first end cover and a second end cover opposite to each other. Either or both of a surface of the first end cover and a surface of the second end cover is provided with a guiding rail 12. One end of the infusion clip 2 is arranged on the guiding rail 12, and the other end of the infusion clip 2 is configured to position an infusion assembly. The infusion assembly is used for infusion to the patient, and, in some embodiments, includes an infusion bag (not shown in the figure) and at least a part of an infusion catheter 10.

In an embodiment, the end of the infusion clip 2 is slidable along the guiding rail. In the CT device, the CT gantry 1 is also used as an infusion stand, and the guiding rail 12 is arranged on the CT gantry 1, so that the infusion assembly can be hung adjacent to the gantry aperture of the CT device, and the infusion assembly fixed to the CT gantry 1 will not interfere with other devices, simplifying the overall structure. In addition, since the infusion clip 2 for fixing the infusion assembly is slidably connected to the guiding rail 12, when a scanning bed 20 moves, a length of the infusion catheter 10 of the infusion assembly outside the guiding rail 12 can be adjusted by moving the infusion clip 2 along the guiding rail 12, so that the infusion catheter 10 will not be wound, stacked or stretched between the scanning bed 20 and the CT gantry 1, so as to meet the infusion requirements of patients in different scanning scenarios.

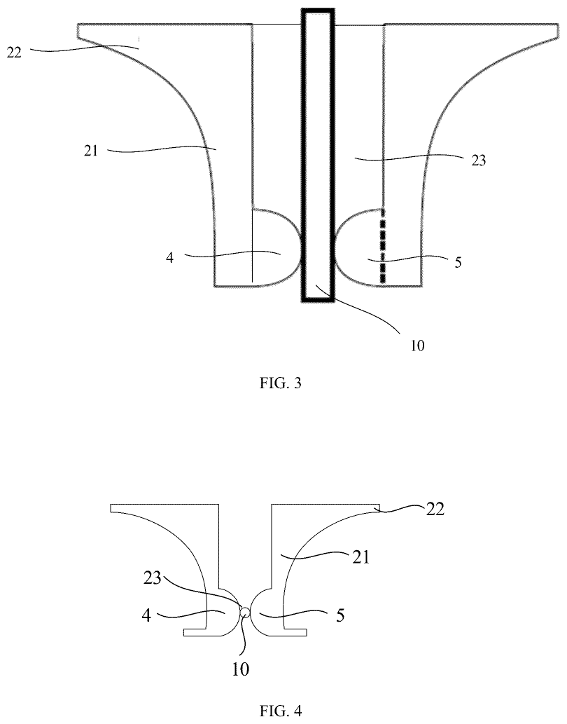

Specifically, referring to FIGS. 1 and 3, the infusion assembly includes the infusion bag and the infusion catheter 10, and the CT device further includes a hook 11 connected to an upper portion of the CT gantry 1. The infusion bag is hung on the hook 11. The infusion catheter 10 is positioned on the infusion clip 2. Since the infusion bag is hung on the hook 11, and the infusion catheter 10 is positioned on the infusion clip 2, the load carried by the infusion clip 2 can be reduced. When the infusion clip 2 moves, the infusion clip 2 only bears the infusion catheter 10 without bearing the infusion bag, which can reduce the weight during transportation and allows for smoother sliding of the infusion clip 2 along the guiding rail 12.

In an embodiment, the infusion catheter 10 is fixed to the infusion clip 2, and the infusion catheter 10 moves along with the infusion clip 2. In another embodiment, the infusion catheter 10 is movable connected to the infusion catheter 10. That is, the infusion catheter 10 can be pulled back and forth relative to the infusion clip 2. In this way, more portions of the infusion catheter 10 can be retracted and released to better meet the infusion requirements of patients in different scanning scenarios. In an embodiment, the infusion clip 2 is provided with a clamping portion, which is configured to clamp the infusion catheter 10 on the infusion clip 2, so that the infusion catheter 10 and the infusion clip 2 cannot move relative to each other, and release the infusion catheter 10, so that the infusion catheter 10 and the infusion clip 2 can move relative to each other. A user can clamp the infusion catheter 10 or release the infusion catheter 10 through the clamping portion as required.

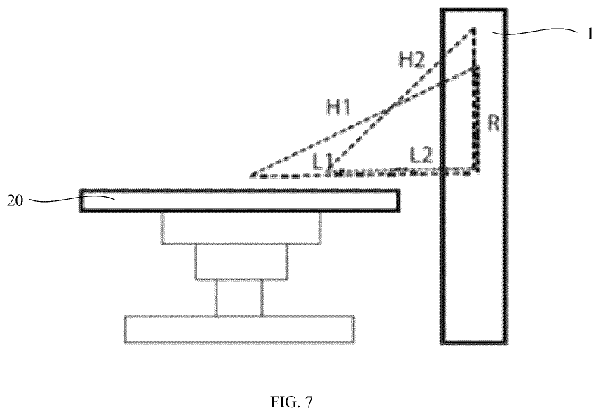

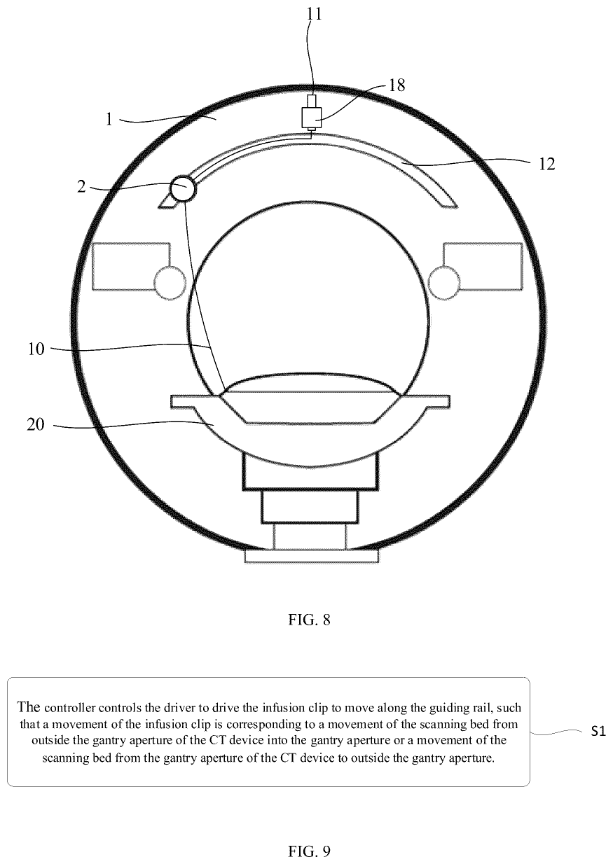

Referring to FIG. 7, in one embodiment, the hook 11 is fixedly to an upper end of the CT gantry 1, for example, the hook 11 is fixedly located above the guiding rail 12. The infusion bag 19 is fixed to the hook 11, one end of the infusion catheter 12 is connected to the infusion bag 19, and the other end of the infusion catheter 12 is located at the patient's inspection portion after passing through the guiding rail 12 and the infusion clip 2. In another embodiment, the hook 11 is slidably provided on the guiding rail 12. When the hook 11 slides along the guiding rail 12 away from the infusion clip 2, the infusion clip 2 is driven by the infusion bag on the hook 11 to along the guiding rail 12, and/or the infusion catheter 10 is driven by the infusion bag on the hook 11 to move relative the infusion clip 2, so as to adjust a length of the infusion catheter 10 on a side of the infusion clip 2 away from the infusion bag.

As shown in FIG. 1, the guiding rail 12 is an arc-shaped structure which is concave downward. The guiding rail 12 with the arc shape is beneficial to cooperate with the CT gantry 1, and a relatively long guiding rail 12 can be arranged on the CT gantry 1, so that a moving distance of the infusion flip 2 on the guiding rail 12 can be increased. In some embodiments, the arc center of the arc-shaped guiding rail 12 is co-centered with the gantry aperture. In another embodiment, in order to facilitate the manufacture of the guiding rail 12, the guiding rail 12 may be a straight structure.

In an embodiment, the upper portions of the first end cover and the second end cover of the CT gantry 1 are each provided with the hook 11, the guiding rail 12, and the infusion clip 2, so that doctors or patients can hang the infusion bag and the infusion catheter 10 adjacent to the patients according to different scanning scenes.

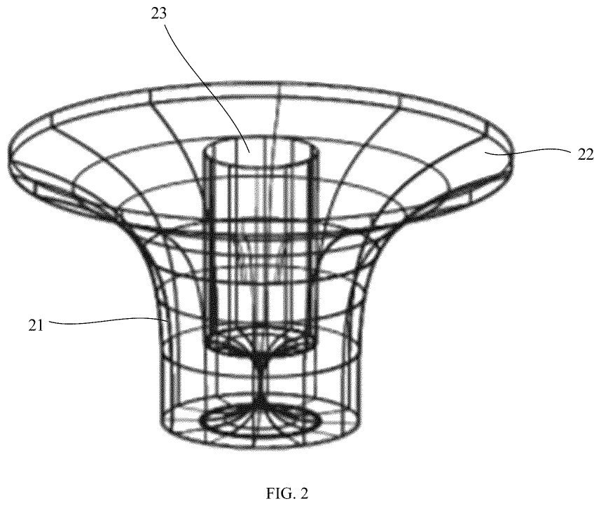

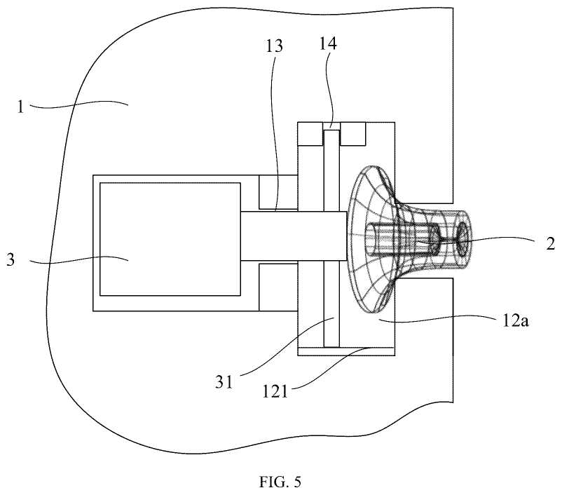

In the present embodiment, as shown in FIGS. 1 and 5, the guiding rail 12 is a locking groove 12a defined by the CT gantry 1. The CT gantry 1 includes two flanges extending toward and spaced from each other, thereby defining opposite edges of the locking groove 12a, and the two flanges are configured to limit the position of the transfusion clip 2. The locking groove 12a is recessed into the CT gantry 1, which occupies less space and has a more compact structure than a protruding guiding rail. The locking groove 12a is also convenient for the infusion clip 2 to be mounted in the locking groove 12a for easy disassembly and assembly. In other embodiments, the guiding rail 12 may be configured as a protruding track or other mechanical structures with guiding functions. As shown in FIG. 2 and FIG. 3, the infusion clip 2 includes a main body 21 and a collar portion 22 located at an end of the main body 21. The collar portion 22 is locked in the locking groove 12a. The main body 21 includes a clamping hole 23 configured to allow the infusion catheter 10 to extend through and to be fixed in the clamping hole 23. In the present embodiment, the clamping hole 23 extends along an axial direction of the main body 21, so that the locking groove 12a can accommodate a part of the infusion catheter 10, so as to neatly arranging the infusion catheter 10. Referring to FIG. 4, in another embodiment, the clamping hole 23 can otherwise extend along a radial direction of the main body 21. The clamping hole 23 can extend along other directions, which is not limited herein.

In order to facilitate an adjustment of an arrangement direction of the infusion catheter 10, an end of the infusion clip 2 adjacent to the guiding rail 12 is rotatably connected to the guiding rail 12, so that the infusion clip 2 can be rotated around the axial direction of the main body 21 to meet the requirement of adjusting the arrangement direction of the infusion catheter 10.

In order to facilitate the assembly, disassembly, and rotation of the infusion clip 2, the infusion clip 2 is made of elastic material, which is also convenient for the infusion catheter 10 to extend therethrough.

In order to improve the fixing effect, a plurality of infusion clips 2 are provided, and the plurality of infusion clips 2 are arranged on the guiding rail 12.

As shown in FIG. 5, the CT device further includes a controller (not shown in the figure) and a driver 3, such as a stepper motor. The stepper motor 3 is axially connected to the infusion clip 2. The controller is electrically connected to the stepper motor 3 and is configured to control the stepper motor 3 to drive the infusion clip 2 to move along the guiding rail 12. The controller controls the stepper motor 3 to rotate, upon which the infusion clip 2 is driven to move along the guiding rail 12, so as to achieve the precise retraction and release of the infusion catheter 10. Since the patient's inspection portion can be known in advance, information of the patient's inspection portion can be input into the controller in advance. When the controller controls the scanning bed 20 to move, the controller also controls the stepper motor 3 to rotate, so that retraction and release of the infusion catheter 10 can be in correspondence with the movement of the scanning bed 20, thereby achieving an automatic adjustment of the infusion catheter 10. In the present embodiment, the stepper motor 3 is axially connected to the lowest infusion clip 2 in the plurality of infusion clips 2. When the stepper motor 3 drives the lowest infusion clip 2 to move, other infusion clips 2 will also be pushed or pulled through the infusion catheter 10 to move.

Specifically, as shown in FIG. 5, the CT device further includes a rack 121 and a pinion 31. The rack 121 is fixed in the locking groove 12a and extends along the locking groove 12a. The pinion 31 is mounted to an output shaft of the stepper motor 3, and the pinion 31 is engaged with the rack 121. When the stepper motor 3 rotates, the pinion 31 is driven to roll on the rack 121, driving the stepper motor 3 to move along the locking groove 12a. Since the stepper motor 3 is axially connected to the infusion clip 2, when the stepper motor 3 moves, the infusion clip 2 is driven to move accordingly. Since the infusion catheter 10 is positioned on the infusion clip 2, when the infusion clip 2 moves along the guiding rail 12, the infusion catheter 10 moves along with the infusion clip 2, so as to adjust the tightness of the infusion catheter 10 between the infusion clip 2 and the infusion site of the patient, or the infusion clip 2 moves relative to the infusion catheter 10, and a portion of the infusion catheter 10 bore on the infusion clip 2 changes accordingly, so that a length of the infusion catheter 10 between the infusion site of the patient and the infusion clip 2 is adjusted to achieve the retraction and the release of the infusion catheter 10. The transmission mode of the pinion 31 and the rack 121 is convenient for the infusion clip 2 to move back and forth. A side of the locking groove 12a adjacent to the stepper motor 3 is provided with a sliding groove 13. The output shaft of the stepper motor 3 extends through the sliding groove 13 and is slidable in the sliding groove 13. The sliding groove 13 is configured to limit the position of the output shaft of the stepper motor 3. Furthermore, a side of the locking groove 12a opposite to the rack 121 is provided with a limiting groove 14. An edge of the pinion 31 is protruded into the limiting groove 14 and the axial position of the pinion 31 is limited by the limiting groove 14, so that the pinion rotates smoothly and the pinion 31 is prevented from being skewed and stuck. The stepper motor 3 is mounted and positioned through the sliding groove 13 and the limiting groove 14 located on peripheral sides of the output shaft, so as to ensure that the pinion 31 is engaged with the rack 121 and rolls along the rack 121. The sliding groove 13 is formed by two first limiting members that are spaced apart, and the two first limiting members are detachably connected to the CT gantry 1, so as to mount the stepper motor 3 and the output shaft of the stepper motor 3. The limiting groove 14 is formed by two second limiting members that are spaced apart, and the two second limiting members are detachably connected to the CT gantry 1, so as to facilitate the mounting of the pinion 31.

As shown in FIG. 3, the CT device further includes a pressure sensor 4, which is mounted in the clamping hole 23 and in contact with the infusion catheter 10. The pressure sensor 4 is configured to monitor a pressure in the infusion catheter 10. The pressure sensor 4 can be electrically connected to a display device which is configured to display a pressure value, or the pressure sensor 4 can be electrically connected to an alarm device. When the pressure value in the infusion catheter 10 exceeds a threshold, the pressure sensor 4 displays the pressure value through the display device or gives an alarm through the alarm device, as an early warning to alert the doctor and the patient. Especially when contrast medium or other medicinal liquid spills out from a blood vessel, the pressure in the infusion catheter 10 increases, and the pressure sensor 4 can provide an early warning.

As shown in FIG. 3, the CT device further includes a flow rate sensor 5, which is mounted in the clamping hole 23 and in contact with the infusion catheter 10. The flow rate sensor 5 is configured to monitor a flow rate of liquid in the infusion catheter 10. It should be noted herein that the flow rate sensor 5 and the pressure sensor 4 are in contact with the infusion catheter 10, and in a case of the infusion catheter 10 being movably provided on the infusion clip 2, it is still satisfied that the infusion clip 2 can move relative to the infusion catheter 10. The flow rate sensor 5 can detect a flow rate of medicinal solution in the infusion catheter 10 in real time, and the flow rate of medicinal solution can provide a basis for the doctor to evaluate a status of the patient in real time. Especially for patients with poor heart function, the status of the patient can be evaluated in real time through real-time detection of the flow rate of the medicinal solution. In the present embodiment, the flow rate sensor 5 is an ultrasonic sensor. The principle of the ultrasonic sensor is based on the Doppler method, which utilizes the reflection of sound waves by a medium to change the frequency of the sound wave. This frequency shift occurs when there is relative motion between the sound source and the medium receiving the sound wave. In other embodiments, the flow rate sensor 5 can be other sensors capable of monitoring the flow rate of liquid. In FIG. 3, only a part of the infusion catheter 10 inside the infusion clip 2 is shown.

In other embodiments, only one of the pressure sensor 4 and the flow rate sensor 5 may be provided.

In other embodiments, the pressure sensor 4 and the flow rate sensor 5 can also be electrically connected to the controller, and the controller receives monitoring values of the pressure sensor 4 and the flow rate sensor 5. When the monitoring value of the pressure sensor 4 and/or the flow rate sensor 5 exceeds a preset value, the controller controls the alarm device to give an alarm as an early warning.

As shown in FIG. 9, a method for controlling a CT device is provided, the method includes following S1.
S1, the controller controls the stepper motor 3 to rotate, thereby driving the infusion clip 2 to move along the guiding rail 12, such that a movement of the infusion clip 2 is corresponding to a movement of the scanning bed 20 from outside the gantry aperture of the CT device into the gantry aperture or a movement of the scanning bed 20 from the gantry aperture of the CT device to outside the gantry aperture.

In the present embodiment, when the scanning bed 20 moves from the outside towards the gantry aperture of the CT device to reach the position of a laser light of the CT device, the infusion site of the patient on the scanning bed 20 gradually approaches the lowest end of the guiding rail 12, and thus the length of the infusion catheter 10 will become redundant. During this period, the controller controls the stepper motor 3 to rotate in a first direction, thereby driving the infusion clip 2 to move down along the guiding rail 12, so as to retract the redundant infusion catheter 10 between the patient's infusion site and the CT gantry 1 in the guiding rail 12, preventing the infusion catheter 10 from being twisted and messy. When the scanning bed 20 continues to move from the position of the laser light toward the gantry aperture, the infusion site of the patient on the scanning bed 20 gradually departs from the lowest end of the guiding rail 12, and the controller controls the stepper motor 3 to rotate in a second direction opposite to the first direction, thereby driving the infusion clip 2 to move upward along the guiding rail 12, so as to release the infusion catheter 10 to meet the distance requirement between the patient's infusion site and the CT gantry 1, and the infusion catheter 10 is prevented from being tightened or falling off, which causes safety hazards.

Conversely, when the scanning bed 20 moves from inside the gantry aperture of the CT device, passes the laser light, and gradually outside the gantry aperture, the stepper motor 3 causes the infusion clip 2 to first retract and then release the infusion catheter 10.

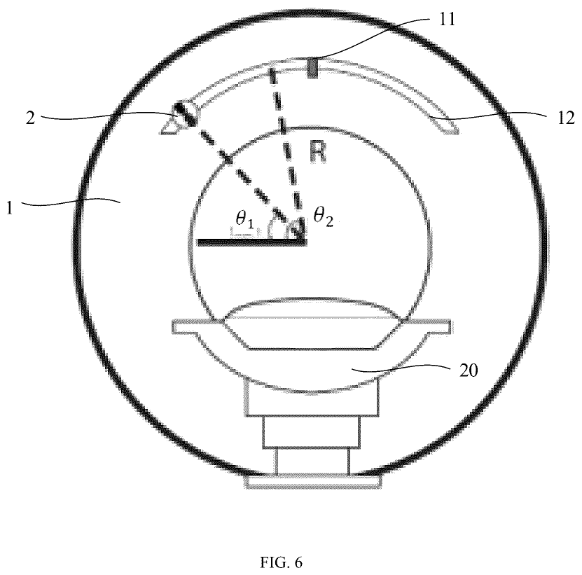

In an embodiment, the controller is further configured to calculate a relationship between a releasing or retracting length of the infusion catheter 10 and a moving angle of the infusion clip 2 on the guiding rail 12. For example, when the scanning bed 20 moves from the outside to the gantry aperture of the CT device, the required length of the infusion catheter 10 is reduced, and the infusion clip 2 may be driven by the stepper motor 3 to descend along the arc-shaped guiding rail 12, so as to reduce a distance between the patient's infusion site and the infusion clip 2. As shown in FIG. 6 and FIG. 7, a formula for calculating the relationship between the releasing or retracting length of the infusion catheter 10 and the moving angle of the infusion clip 2 on the guiding rail 12 is as follows. The patient's injection site, a center of the gantry aperture of the CT gantry 1, and the infusion clip 2 form a spatial triangle. The releasing or retracting length Δ1 of the infusion catheter 10 is calculated according to the formula (1).

Δ1=|\sqrt{R²+L₁²}−\sqrt{R²+L₂²}| (1)

In the formula (1), Δ1 is the releasing or retracting length of the infusion catheter 10. R is a radius of the arc-shaped guiding rail 12. L₁ is a distance between the patient's infusion site at an initial position and the center of the gantry aperture of the CT gantry 1, which may be monitored by a monitoring member. L₂ is a real-time distance between a position of the patient's infusion site moving with the scanning bed 20 and the center of the gantry aperture of the CT gantry 1, which may be also monitored by the monitoring member. H₁ is a distance between the patient's infusion site at the initial position and the infusion clip 2, and is a real-time distance between the position of the patient's infusion site moving with the scanning bed 20 and the infusion clip 2. The difference between and is equal to the releasing or retracting length of the infusion catheter 10.

According to the following formula (2), the moving angle θ of the infusion clip 2 moving along the guiding rail 12 relative to the center of the gantry aperture of the CT gantry 1 can be calculated.

θ=180*Δ1/(π*R) (2)

As shown in FIG. 6, θ₁ is an angle between a line connecting the infusion clip 2 to the center of the gantry aperture of the CT gantry 1 and a horizontal plane when the patient's infusion site is located at the initial position, and θ₂ is a real-time angle between a line connecting the infusion clip 2 to the center of the gantry aperture of the CT gantry 1 and the horizontal plane during the movement of the patient's infusion site with the scanning bed 20. The moving angle θ of the infusion clip 2 is equal to a difference between the angle θ₁ and the angle θ₂, which is also an angle corresponding to the distance the infusion clip 2 needs to be moved on the guiding rail 12. The controller is further configured to control the stepper motor 3 to rotate a number of revolutions corresponding to the moving angle θ of the infusion clip 2, and then the position of the infusion clip 2 can be adjusted dynamically to achieve automatic adjustment of the infusion catheter 10.

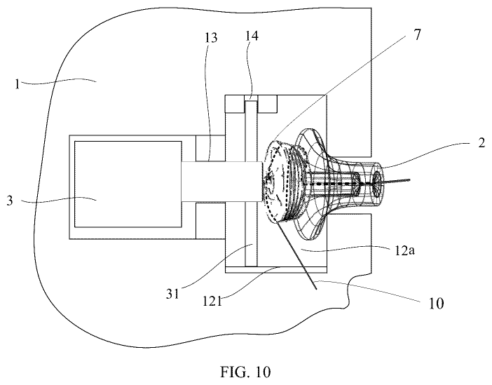

Referring to FIG. 10, in another embodiment, compared with the CT device shown in FIG. 5, the CT device in this embodiment further includes a rotating wheel 7. The infusion catheter 10 extends through the infusion clip 2 and is partially wound on an outer periphery of the rotating wheel 7. The rotating wheel 7 is configured to rotate when the infusion clip 2 is located at the end of the guiding rail 12, so that the infusion catheter 10 is wound around the outer periphery of the rotating wheel 7 or the infusion catheter 10 wound around the rotating wheel 7 is released through the rotating wheel 7.

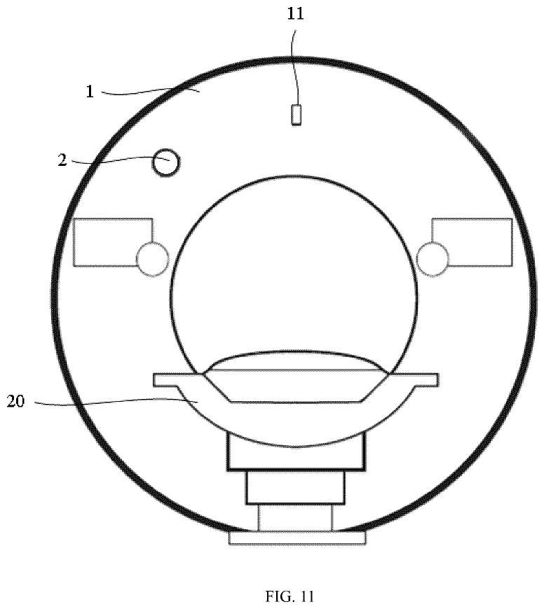

Specifically, the rotating wheel 7 is provided between the infusion clip 2 and the stepper motor 3 and is located in the guiding rail 12. The output shaft of the stepper motor 3 is detachably connected to the rotating wheel 7. When the stepper motor 3 drives the infusion clip 2 to move, the output shaft of the stepper motor 3 is disengaged from the rotating wheel 7, so that the rotating wheel 7 moves along the guiding rail 12 without rotation. When the infusion clip 2 is located at the end of the guiding rail 12, the output shaft of the stepper motor 3 is engaged with the rotating wheel 7, so as to drive the rotating wheel 7 to rotate, so that the infusion catheter 10 is wound around the outer periphery of the rotating wheel 7 or the infusion catheter 10 wound around the rotating wheel 7 is released through the rotating wheel 7. In an embodiment, an outer periphery of the middle portion of the rotating wheel 7 is recessed. Referring to FIG. 11, in an embodiment, no guiding rail 12 is provided on the CT gantry 1, the infusion clamp 2 is connected to the CT frame 1. The retraction and the release of the infusion catheter 10 is achieved only by the stepper motor 3 driving the rotating wheel 7 to rotate to enable the infusion catheter 10 to be wound around the outer periphery of the rotating wheel 7 and the infusion catheter 10 wound around the rotating wheel 7 to be released through the rotating wheel 7.

In an embodiment, the output shaft of the stepper motor 3 includes a first shaft away from a motor body of the stepper motor 3 and a second shaft smoothly connected to the first shaft. A diameter of the second shaft is greater than a diameter of the first shaft, and a size of a hole of the rotating wheel 7 corresponds to the diameter of the second shaft. When the infusion clip 2 is not located at the end of the guiding rail 12, the rotating wheel 7 is sleeve on the first shaft, and when the infusion clip 2 moves along the guiding rail 12, the rotating wheel 7 also moves along the guiding rail 12 without rotating. When the infusion clip 2 is located at the end of the guiding rail 12, the second shaft of the stepper motor 3 is inserted into the rotating wheel 7 to drive the rotating wheel 7 to rotate, so that the infusion catheter 10 is wound around the outer periphery of the rotating wheel 7 or the infusion catheter 10 wound around the rotating wheel 7 is released through the rotating wheel 7. Thereby the retraction and the release of the relatively long infusion catheter 10 is achieved.

When the infusion clip 2 moves to the end of the guiding rail 12 far away from the infusion bag, the retracting length of the infusion catheter 10 is the maximum, at this time, the retracting length of the infusion catheter 10 L_{end} is calculated according to the formula (3)

L_{end}=πRθₘₐₓ/180 (3)

In the formula (3), θₘₐₓ is an angle between a straight line connecting the infusion bag 11 to the center of the gantry aperture of the CT gantry 1 and a straight line connecting the end of the guiding rail 12 to the center of the gantry aperture of the CT gantry 1.

According to a change trend between the moving distance of the bed and the retracting length of the infusion catheter 10, when the portion of the infusion catheter 10 on patient's injection site is directly below the infusion bag 11, that is, directly below the hook 11, the retracting length of the infusion catheter 10 is the maximum. The retracting length of the infusion catheter 10 is defined as Lₘₐₓ.

In order to enable the CT device to automatically retract the infusion catheter 10 when the retracting length Lₘₐₓ is greater than the retracting length L_{edge}, the controller is further configured to control the stepper motor to drive the rotating wheel 7 to rotate when the infusion clip 2 moves to the end of the guiding rail, so that more parts of the infusion catheter 10 are wound around the outer periphery of the rotating wheel 7 or more parts of the infusion catheter 10 wound around the outer periphery of the rotating wheel 7 are released. Thereby the retraction and the release of the relatively long infusion catheter 10 is achieved.

When the infusion clip 2 is located at the end of the guiding rail 12, the length L_{2end} between the patient's infusion site and the center of the CT gantry 1 is calculated according to the formula (4).

$${L}_{2 end} = \sqrt{{( \sqrt{{R}^{2} + {L}_{1}^{2}} - π R {θ}_{max} / 180 )}^{2} - {R}^{2}} \qquad ( 4 )$$

Since the rotating wheel 7 rotate only when the transfusion clip 2 is located at the end of the guiding rail 12, L₂ in the formula (4) is equivalent to a distance between the patient's infusion site at an initial position and the center of the gantry aperture of the CT gantry 1 during the rotation of the rotating wheel 7. Thus, similar to the formula (1), the controller is further configured to calculate the retracting or releasing length of the infusion catheter 10 realized by the rotation of the rotating wheel 7 according to the following formula (5).

Δ1r=|\sqrt{R²+L_{2end}²}−\sqrt{R²+L₂²}| (5)

The controller is further configured to calculate a rotation angle θᵣ that the rotating wheel 7 needs to rotate according to the following formula (6).

θᵣ=180Δ1r/(πR_{wheel}) (6)

In the formula (6), R_{wheel} is a radius of the rotating wheel 7.

The controller is further configured to control the stepper motor to rotate a number of revolutions corresponding to the rotation angle θᵣ that the rotating wheel 7 needs to rotate when the infusion clip is located at the end of the guiding rail 12, and then further dynamically retract or release the infusion catheter 10 to achieve automatic adjustment of the infusion catheter 10.

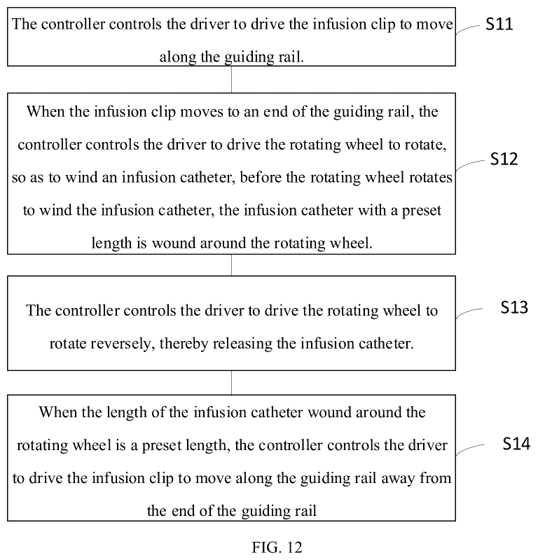

As shown in FIG. 12, a method for controlling a CT device is provided, the method includes following steps.
Step S11, the controller controls the driver 3 to drive the infusion clip 2 to move along the guiding rail 12.
Step S12, when the infusion clip 2 moves to an end of the guiding rail, the controller controls the driver 3 to drive the rotating wheel 7 to rotate, so as to wind an infusion catheter 10, before the rotating wheel 7 rotates to wind the infusion catheter 10, the infusion catheter 10 with a preset length is wound around the rotating wheel 7.

The method for controlling the CT device further includes the following steps.
Step S13, the controller controls the driver 3 to drive the rotating wheel 7 to rotate reversely, thereby releasing the infusion catheter 10.
Step S14, when the length of the infusion catheter 10 wound around the rotating wheel 7 is a preset length, the controller controls the driver 3 to drive the infusion clip 2 to move along the guiding rail 12 away from the end of the guiding rail 12.

The foregoing descriptions are merely specific embodiments of the present disclosure, but are not intended to limit the protection scope of the present disclosure. Any variation or replacement readily figured out by a person skilled in the art within the technical scope disclosed in the present disclosure shall all fall within the protection scope of the present disclosure.

What is claimed is:

1. A CT device, comprising:
a CT gantry; and
at least one infusion clip, each infusion clip being connected to the CT gantry and configured to position an infusion assembly.

2. The CT device according to claim 1, further comprising:
a hook, connected to an upper portion of the CT gantry, wherein the infusion assembly comprises an infusion bag and an infusion catheter, the hook is configured to hang the infusion bag, and the infusion clip is configured to carry the infusion catheter.

3. The CT device according to claim 2, wherein the infusion catheter is fixed to the infusion clip or movably provided on the infusion clip.

4. The CT device according to claim 2, wherein the hook is fixed to the CT gantry or slidably connected to the CT gantry.

5. The CT device according to claim 2, wherein the CT gantry is provided with a locking groove, the infusion clip comprises a main body and a collar portion located at an end of the main body, the collar portion is locked in the locking groove, the main body defines a clamping hole, and the clamping hole is configured to allow the infusion catheter to extend through and to be carried in the clamping hole.

6. The CT device according to claim 5, further comprising:
a pressure sensor mounted in the clamping hole and abutting against the infusion catheter, and the pressure sensor being configured to monitor a pressure in the infusion catheter.

7. The CT device according to claim 5, further comprising:
a flow rate sensor mounted in the clamping hole and abutting against the infusion catheter, and the flow rate sensor being configured to monitor a flow rate of liquid in the infusion catheter.

8. The CT device according to claim 1, further comprising:
a driver axially connected to the infusion clip; and
a controller electrically connected to the driver, wherein the CT gantry is provided with a guiding rail, the controller is configured to control the driver to drive the infusion clip to move along the guiding rail.

9. The CT device according to claim 8, further comprising:
a rack is disposed adjacent to and parallel with the guiding rail; and
a pinion mounted to an output shaft of the driver and engaged with the rack.

10. The CT device according to claim 1, wherein the CT gantry is provided with a guiding rail, at least one infusion clip infusion clip is connected to guiding rail, the guiding rail is in an arc-shaped structure which is concave downward, or the guiding rail is arranged in a straight structure.

11. The CT device according to claim 1, wherein the infusion clip is rotatably connected to the CT gantry.

12. The CT device according to claim 1, wherein a plurality of infusion clips are provided on the CT gantry.

13. The CT device according to claim 1, wherein the CT gantry comprises a first end cover and a second end cover opposite to each other, either or both of a surface of the first end cover and a surface of the second end cover is provided with a guiding rail, at least one infusion clip infusion clip is connected to the guiding rail.

14. The CT device according to claim 3, further comprising a rotating wheel, wherein the infusion catheter extends through the infusion clip and is partially wound on an outer periphery of the rotating wheel, the rotating wheel is configured to enable the transfusion catheter to be wound on the rotating wheel during rotation.

15. The CT device according to claim 14, wherein the CT gantry is provided with a guiding rail, the rotating wheel rotates when the infusion clip is located at the end of the guiding rail.

16. The CT device according to claim 14, further comprising a steeper motor, wherein the rotating wheel is provided between the infusion clip and the stepper motor, an output shaft of the stepper motor is detachably connected to the rotating wheel.

17. A method for controlling the CT device according to claim 1, comprising:
driving the infusion clip to move along a guiding rail provided on the CT device, such that a movement of the infusion clip is corresponding to a movement of a scanning bed from outside a gantry aperture of the CT device into the gantry aperture or a movement of the scanning bed from the gantry aperture to outside the gantry aperture.

18. The method for controlling the CT device according to claim 17, further comprising:
driving a rotating wheel to rotate to wind an infusion catheter when the infusion clip moves to an end of the guiding rail, wherein before the rotating wheel rotates to wind the infusion catheter, the infusion catheter with a preset length is wound around the rotating wheel.

19. The method for controlling the CT device according to claim 18, further comprising:
driving the rotating wheel to rotate reversely to release infusion catheter.

20. The method for controlling the CT device according to claim 19, further comprising:
driving the infusion clip to move along the guiding rail away from the end of the guiding rail when a length of the infusion catheter wound on the rotating wheel is the preset length.
